(19) United States
(12) Patent Application Publication
(10) Pub. No.: US 2022/0347491 A1
(43) Pub. Date: Nov. 3, 2022

(54) SYSTEMS AND METHODS OF ADAPTIVE RADIOTHERAPY WITH CONVENTIONAL LINEAR PARTICLE ACCELERATOR (LINAC) RADIOTHERAPY DEVICES

(71) Applicant: Washington University, St. Louis, MO (US)

(72) Inventors: Taeho Kim, St. Louis, MO (US); Matthew Schmidt, St. Louis, MO (US)

(21) Appl. No.: 17/735,586

(22) Filed: May 3, 2022

Related U.S. Application Data
(60) Provisional application No. 63/183,277, filed on May 3, 2021.

(51) Int. Cl. A61N 5/10; G06T 7/00

(52) U.S. Cl. CPC A61N 5/1039; G06T 7/0012; A61N 5/1083; A61N 5/1001

(57) ABSTRACT

A linear particle accelerator (LINAC) radiotherapy system of a subject is provided. The system includes a gantry and an adaptive radiotherapy computing device. The gantry includes a radiation delivery assembly including LINACs and an x-ray imaging assembly, wherein the gantry defines a c-arm. The at least one processor of the adaptive radiotherapy computing device is programmed to receive first images of the subject acquired by an imaging system, and receive second images of the subject acquired by the x-ray imaging assembly, wherein the first images have higher resolutions than the second images. The at least one processor is further programmed to adapt a treatment plan using the second images, wherein the treatment plan was designed based on the first images, and a level of optimization in adapting the treatment plan is adjustable. The at least one processor is also programmed to output the adapted treatment plan.

### CROSS-REFERENCE TO RELATED APPLICATIONS

This patent application claims the benefit of U.S. Provisional Patent Application No. 63/183,277, filed on May 3, 2021, titled “ON-TABLE ADAPTIVE RADIOTHERAPY (ART) WITH CONVENTIONAL LINACS,” the entire contents and disclosures of which are hereby incorporated herein by reference in its entirety.

### BACKGROUND

The field of the disclosure relates generally to radiotherapy, and more particularly, to systems and methods of adaptive radiotherapy with conventional linear particle accelerator (LINAC) devices.

Adaptive radiotherapy using magnetic resonance (MR)-guided adaptive radiotherapy increases overall survival with low toxicity. Conventional LINAC devices, however, do not have the functionality of adaption. Known adaptive radiotherapy systems and methods are disadvantaged in some aspects and improvements are desired.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION

In one aspect, a linear particle accelerator (LINAC) radiotherapy system of a subject is provided. The system includes a gantry and an adaptive radiotherapy computing device. The gantry includes a radiation delivery assembly including LINACs and configured to generate radiation and an X-ray imaging assembly configured to image a subject, wherein the gantry defines a C-arm. The adaptive radiotherapy computing device includes at least one processor in communication with at least one memory device. The at least one processor is programmed to receive first images of the subject acquired by an imaging system, and receive second images of the subject acquired by the X-ray imaging assembly, wherein the first images have higher resolutions than the second images. The at least one processor is further programmed to adapt a treatment plan using the second images, wherein the treatment plan was designed based on the first images, and a level of optimization in adapting the treatment plan is adjustable. The at least one processor is also programmed to output the adapted treatment plan.

In another aspect, an adaptive radiotherapy computing device of a LINAC radiotherapy system is provided. The adaptive radiotherapy computing device includes at least one processor in communication with at least one memory device. The at least one processor is programmed to receive first images of a subject acquired by an imaging system, and receive second images of the subject acquired by an X-ray imaging assembly in a C-arm gantry of a LINAC radiotherapy system, wherein the first images have higher resolutions than the second images. The at least one processor is further programmed to adapt a treatment plan using the second images, wherein the treatment plan was designed based on the first images, and a level of optimization in adapting the treatment plan is adjustable. The at least one processor is also programmed to output the adapted treatment plan.

In one more aspect, a method of adapting radiotherapy on a subject with a LINAC radiotherapy system is provided. The method includes receiving first images of a subject acquired by an imaging system, and receiving second images of the subject acquired by an X-ray imaging assembly in a C-arm gantry of a LINAC radiotherapy system, wherein the first images have higher resolutions than the second images. The method also includes adapting a treatment plan using the second images, wherein the treatment plan was designed based on the first images, wherein adapting a treatment plan further includes adjusting a level of optimization in adapting the treatment plan. The method also includes outputting the adapted treatment plan.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present disclosure will become better understood when the following detailed description is read with reference to the accompanying drawings in which like characters represent like parts throughout the drawings.

FIG. 1A shows the gantry and couch of an exemplary adaptive radiotherapy system.

FIG. 1B is a schematic diagram of the system shown in FIG. 1A.

FIG. 2 is known adaptive radiotherapy system.

FIG. 3 is a flow chart of an exemplary method of adaptive radiotherapy.

FIG. 4. is an exemplary work flow of the system shown in FIG. 1B.

FIG. 5A shows an original plan and predicted plans in a nonadaptive treatment plan.

FIG. 5B shows an original plan and adaptive plans using the systems and methods shown in FIGS. 1A, 1B, 3, and 4.

FIG. 5C shows an original plan and day 1 plans of the predicted plans and adaptive plans.

FIG. 5D shows an original plan and day 2 plans of the predicted plans and adaptive plans.

FIG. 5E shows an original plan and day 3 plans of the predicted plans and adaptive plans.

FIG. 6 is a block diagram of an exemplary computing device.

FIG. 7 is a block diagram of a server computing device.

Unless otherwise indicated, the drawings provided herein are meant to illustrate features of embodiments of the disclosure. These features are believed to be applicable in a wide variety of systems including one or more embodiments of the disclosure. As such, the drawings are not meant to include all conventional features known by those of ordinary skill in the art to be required for the practice of the embodiments disclosed herein.

### DETAILED DESCRIPTION

The disclosure includes systems and methods of adaptive radiotherapy of a subject. As used herein, a subject is a human, an animal, or a phantom, or part of the human, the animal, or the phantom such as an organ or part of an organ.

In radiotherapy, a treatment plan is developed based on an image of the subject. Developing a treatment plan afresh may take 6-8 hours. Radiotherapy treatment is performed in fractions, number of days, or sessions of treatments. During a fraction, the treatment plan for that fraction is delivered. Conventional radiotherapy devices or linear particle accelerator (LINAC) devices (e.g., TrueBeam™ by Varian Medical Systems®) were developed a decade ago and have been install at over 7000 sites in the United States. A conventional radiotherapy device use a LINAC to generate radiation for treatment of diseases. The radiation emitted from the device targets malignant tissue such as tumor for the goal of reducing and eventually removing the tumor. As treatment progresses, the locations and/or sizes of the tumor may change. During the treatment, the tumor may also move. If the tumor or the subject has changed significantly, rendering the treatment plan unusable for treatment, the treatment fraction would need to be cancelled and a new treatment plan would need to be developed. Further, conventional radiotherapy devices do not have the functionalities of adapting the treatment plan based on the anatomy changes of the tumor. As a result, the treatment efficacy is not optimized.

Recently, image-guided adaptive radiotherapy has been developed. In image-guided adaptive radiotherapy, images are acquired on the day of the treatment. Images acquired on the treatment day may be referred to as daily images. The treatment plan is adapted based on the daily image to account for anatomy changes.

There are two known adaptive radiotherapy systems, MR-guided adaptive radiotherapy (MRgART) systems and X-ray guided adaptive radiotherapy systems. MRgART has led to enhanced overall survival with low toxicity because of excellent critical organ visualization, detection of anatomical changes, and corresponding treatment adaptation in an on-table setting. However, reproducing these excellent results in the broad community setting is challenging because MRgART is not widely available, is expensive, and requires specialized expertise (e.g., MR safety), which is not typically found in community radiotherapy practices.

X-ray guided adaptive radiotherapy system, such as Ethos™ by Varian Medical Systems®, emerged into the market about three years ago. Results on survival rates and toxicity from x-ray guided adaptive radiotherapy are not available yet. The known X-ray guided adaptive radiotherapy system uses cone beam computed tomography (CBCT) to acquire daily images and adapts treatment plans based on the daily images, in order to optimize delivery time and accuracy. The known X-ray guided adaptive system is not widely received in clinical settings due to the limited treatment functionalities and inflexible and relatively long treatment time. In radiotherapy, treatment time refers to beam-on time, and starts when the beam such as the X-ray from X-ray imaging assembly 106 is on or emitted. The known X-ray guided therapy system is a less expensive version of the conventional radiotherapy system with simplified gantry and LINAC. The known X-ray guided adaptive system includes a ring-type gantry, which is not capable of non-coplanar plan delivery, limiting dosage optimization and applications. The known X-ray guided therapy system also limits the CBCT image acquisitions to non-gated acquisitions. To reduce the effects of motion, subjects are instructed to hold breath during imaging, which is challenging for subjects with impaired breathing. Further, the known X-ray guided adaptive system is a closed system, where a dedicated therapy treatment planning system including adaption of the treatment plan is included in the radiotherapy system and a fixed patient protocol of daily imaging and adaptive planning is operated for each fraction of the treatment. As a result, treatment using the known X-ray guided adaptive system takes longer than the conventional radiotherapy systems. Currently, less than ten known X-ray guided adaptive radiotherapy systems are installed in the United States.

Systems and methods described herein provide adaptive radiotherapy that overcomes the above-described problems in known adaptive radiotherapy systems. The systems and methods implement adaptive radiotherapy in a conventional radiotherapy system, using the existing modules in the conventional radiotherapy system. Conventional radiotherapy systems are expensive. Implementing adaptive radiotherapy without significant changes to the conventional radiotherapy system provides a cost-effective approach to improve the functionalities of the conventional system without costly changes to the conventional system. Further, systems and methods describe herein provide flexible work flows, adjustable based on the degree of changes in the tumor, where the level of optimization of the treatment plan may be adjusted, thereby minimizing the penalty from increase in treatment time. Moreover, systems and methods described herein are advantageous over known adaptive radiotherapy system because the adapted treatment plan by the known radiotherapy system is restricted to ring-type gantry, resulting in an un-optimized or even inoperable treatment plan for a conventional radiotherapy system.

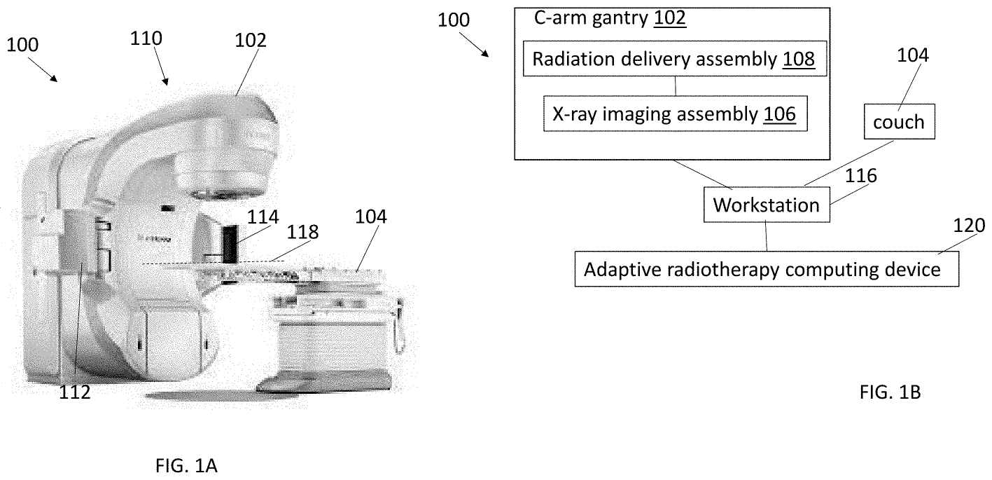

FIGS. 1A and 1B show an exemplary LINAC radiotherapy system 100. FIG. 1A shows a gantry 102 and a couch 104 of system 100. FIG. 1B is a schematic diagram of system 100. In the exemplary embodiment, system 100 includes C-arm gantry 102, where gantry 102 forms into a C-arm 110 or arm 110 in the shape of letter C. Gantry 102 includes an X-ray imaging assembly 106 and a radiation delivery assembly 108. X-ray imaging assembly 106 may be a CBCT imaging assembly. Alternatively, X-ray imaging assembly 106 is a CT imaging assembly. Radiation delivery assembly 108 includes a LINAC and is configured to generation radiation used for therapy. X-ray imaging assembly 106 includes a cone beam emitter 112 and a detector 114. Cone beam emitter 112 emits divergent X-rays that form a cone. Cone beam emitter 112 and detector 114 are positioned opposite from and facing one another such that detector 114 detects cone-beam X-rays absorbed, attenuated, and/or deflected by a subject (not shown). Images the subject are generated by reconstructing the signals detected by detector 114.

In the exemplary embodiment, system 100 also includes a couch 104. During treatment, a subject is lying on couch 104. System 100 further includes a workstation 116 that controls the operation of C-arm gantry 102 and couch 104.

In operation, radiation delivery assembly 108 and X-ray imaging assembly 106 are in C-arm gantry 102 and are positioned approximately 90° from one another. C-arm gantry 102 may be rotated with three degrees of freedom, where the rotation axis 118 around which C-arm gantry 102 rotates may be oriented in an arbitrary 3D angle. Couch 104 may be rotational with one degree of freedom for treatment, rotating 90° to 270° through 0° (or 360°), and with three degrees of freedom of a limited range for subject setup. During treatment, a protocol including a treatment plan is loaded in workstation 116. X-ray imaging assembly 106 acquires images of the subject. The acquired images are used to guide the delivery of radiation towards to target regions such as a tumor in the subject. The treatment plan is adjusted depending on the chronical timing of a fraction in the entire treatment. For example, a later fraction may have a different dose, compared to an earlier fraction, due to the expectation of changes in the tumor from treatment.

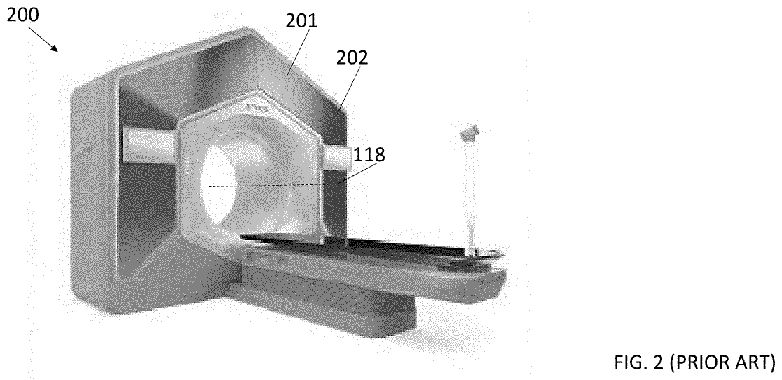

FIG. 2 is a known adaptive LINAC radiotherapy system 200. Compared to system 100, known system 200 includes a ring-type gantry 201 and a nonrotatable couch 104. X-ray imaging assembly and radiation delivery assembly are positioned in a ring 202 of ring-type gantry 201 and may only be rotated around a fixed rotation axis 118. When a subject is positioned on couch 104 and in a ring 202 of ring-type gantry 201, X-ray imaging assembly and radiation delivery assembly may only be rotated around fixed rotation axis 118, producing only axial images of the subject and delivering radiation toward the subject in a coplanar manner. Known system 100 includes an adaptive contouring and planning configured to adapt a treatment plan based on anatomy changes from the planning stage. Adaptation is performed during all fractions of the treatment.

In contrast, system 100 provide images in orientations such as sagittal images, besides 2D or 3D axial images. Images of various orientations provide a better depiction of the subject than axial-only images. System 100 also delivers radiation at optimized doses that conforms to the geometry of the malignant tissue, and limit radiation to sensitive structures such as spinal cord and brain stem. As a result, system 100 provides improved performance in treating types of cancers that are difficult for known system 200 to deliver radiation. System 100 also provides gated functions during treatment of a moving organ such as the lung, where the radiation is timed according to the moving cycles of the organ such that radiation is delivered to the same location in the moving organ, thereby optimizing the efficacy of treatment.

Systems having c-arm gantries have been developed a few decades before and are widely available in clinics. Due to the limited performance, known systems 200 are not widely received, despite an improved performance from adaptation.

Referring back to FIGS. 1A-1B, system 100 also includes an adaptive radiotherapy computing device 120. Adaptive radiotherapy computing device 120 is configured to adapt a treatment plan of the subject. Adaptive radiotherapy computing device 120 may be included in workstation 116 pre-existed in system 100, or may be included in a separate computing device that is in communication with workstation 116, through wired or wireless communication. In one example, adaptive radiotherapy computing device 120 is a server computing device, and may be cloud-based. In some embodiments, adaptive radiotherapy computing device 120 is a separate computing device from workstation 116 and receives images acquired by workstation 116 through a portable storage device, such as a flash drive or a thumb drive.

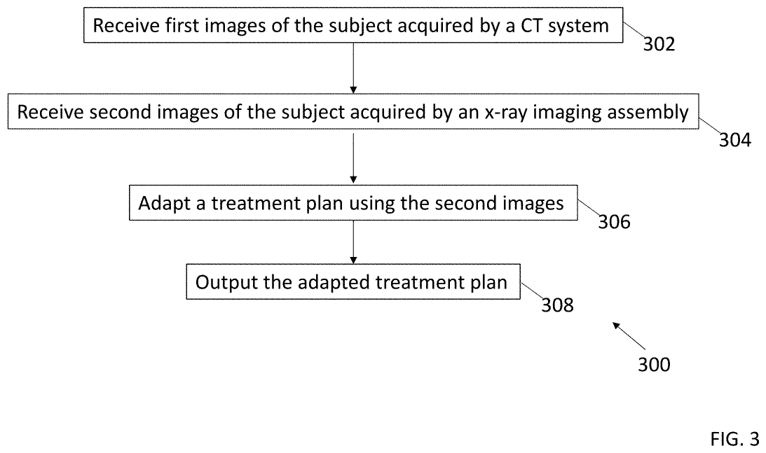

FIG. 3 is a flow chart of an exemplary method 300 of adaptive radiotherapy. Method 300 may be implemented in adaptive radiotherapy computing device 120. In the exemplary embodiment, method 300 includes receiving 302 first images of the subject acquired by an imaging system used for estimating dosage in treatment planning. The imaging system may be a CT system. The CT system is a separate system from X-ray imaging assembly 106 of system 100. Method 300 also includes receiving 304 second images of the subject acquired by the X-ray imaging assembly. The first images acquired by a CT system have higher image resolutions than the second images acquired by X-ray imaging assembly 106. First images were acquired before the treatment and during the stage of treatment planning. First images are used to generate radiation treatment plan. Because the first and second images are acquired by different imaging systems and at different time, the two sets of images do not align with one another. Method 300 further includes adapting 306 the treatment plan using the second images. Adapting 306 may include adaptive contouring and/or adaptive planning. After an adaptive treatment plan is generated, quality assurance of the adapted treatment plan may be performed. Quality assurance may be performed on the deliverability of the adapted treatment plan, to examine whether delivery of the treatment in the plan is within the functionalities of system 100. If the treatment plan is outside the functionalities of system 100, the treatment plan is adjusted. Quality assurance may be performed on the achievability of the adapted plan, where whether the treatment plan will achieve the target effect on malignant tissue is evaluated. If treatment plan will likely not achieve the target effect, the treatment plan is adjusted. Moreover, method 300 includes outputting 308 the adapted treatment plan. Method 300 also includes treating the subject via radiation delivery assembly 108 according to the adapted treatment plan. Adaption of the treatment plan and treatment according to the adapted treatment plan are accomplished while the subject is lying on couch 104. The adapted treatment plan accounts for the differences between the imaging system used during the planning stage and X-ray imaging assembly 106 of radiotherapy system 100 during the treatment stage. The adapted treatment plan also accounts for changes to the anatomies of the malignant tissue and the subject from the planning stage to the treatment stage and among different fractions in the treatment stage. Replanning would not be needed even if the anatomy changes are significant. As a result, the treatment efficacy is optimized and inconvenience to the subject is reduced.

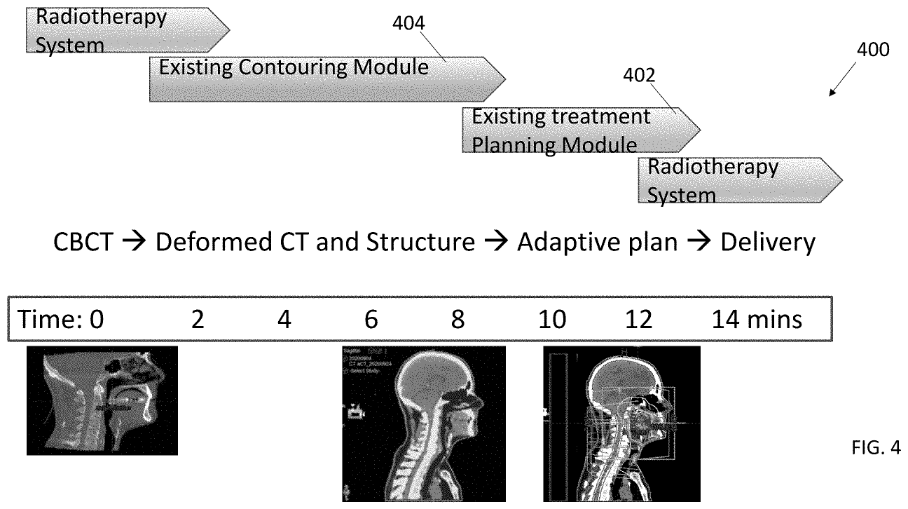

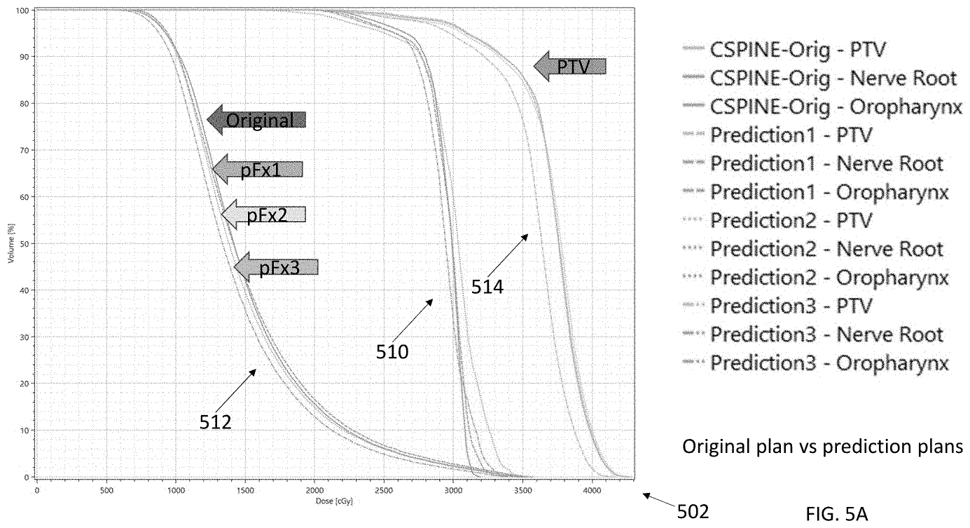

FIG. 4 is an exemplary work flow 400 of adaptive radiotherapy, implementing method 300 using existing functions and/or modules in system 100. The whole process may take up to 15 minutes, and on average takes 3-4 minutes. This duration of time is reasonable for a subject who is lying on couch 104 during the treatment. In an existing work flow, CBCT images are acquired by X-ray imaging assembly 106 of radiotherapy system 100. Contours of target regions are constructed based on the CBCT images using existing contouring module. A treatment plan generated by an existing treatment planning module 402 is used to deliver radiation toward the targeted regions contoured by the existing contouring module. In the exemplary embodiment, CT images acquired by a CT system are registered onto CBCT images acquired by X-ray imaging assembly 106, aligning CT images with the CBCT images. Registration may include transformation of the CT images such as rigid transformation, e.g., translation and rotation, and non-rigid transformation, e.g., deformation. Registration may take 2 minutes. The registered CT images are input into existing contouring module 404. During this procedure, subject-specific adaptive CT and structural contours are constructed with existing contouring module 404. The accuracy of adaptive CT and structural contours are evaluated and improved using anatomical key structures.

In the exemplary embodiment, adaptive radiotherapy planning is performed. In parallel with adaptive CT and structure generation, adaptive treatment plan is developed in existing treatment planning module 402. The procedure may have two components for use: initial plan preparation and rapid adaptive plan generation. The generation of initial planning criteria may take approximately 30 seconds in addition to the adaptive planning process. During initial plan preparation, the treatment plan is modified based on the changes in contours and structures detected by existing contouring module 404. Initial planning will act as a standardized component to provide a stable function of the on-table adaptive plan quality. On-table as used herein refers to the adaptation is implemented while the subject is lying on couch 104. Therefore, the time spent on adaptation is typically less than half an hour for on-table adaptive planning. Rapid adaptive planning is a flexible component in adaptive planning. Script-based adaptive planning and/or knowledge-based adaptive planning is included in rapid adaptive planning in the on-table setting. Script-based adaptive planning uses scripts compatible with an existing system 100, limiting modification to system 100 and reducing costs and time in retrofitting and/or upgrading.

In the exemplary embodiment, prior knowledge is used in adapting treatment plans. Prior knowledge is a set of relationships between radiation delivered and outcome of the malignant tissue. Prior knowledge may be a set of mathematical functions of the geometrical properties of radiation and the treatment plan. Prior knowledge is not used in known X-ray guided adaptive system 200 because including prior knowledge would increase the already relatively long duration of a treatment fraction. In some embodiments, options on levels of optimization are included in adapting 306 a treatment plan. The levels of optimizations may include whether to include prior knowledge or not, the amount of prior knowledge to include, and the number of repetitions in optimization. If the anatomy of the malignant tissue or the subject has not significantly changed, the level of optimization may be low and the optimization time may last as low as two minutes. If the anatomy of the malignant tissue or the subject has significantly changed, a full optimization may be applied and the optimization time may last 15 minutes. This amount of time is still relatively minimal, compared to a conventional system that does not include the systems and methods described herein, where the treatment fraction would need to be cancelled if the malignant tissue or the subject has significantly changed.

In the exemplary embodiment, the on-table adaptation evaluation with virtual clinical studies is conducted. The on-table adaptation procedure is accomplished through combining the components developed in generation of adaptive CT, structures, and adaptation plans. Then, the on-table adaptation procedure is evaluated using an existing clinical X-ray imaging-guided radiotherapy system (e.g., TrueBeam). Overall clinical value of the proposed tools are evaluated with a physical phantom study and a virtual clinical trial using an existing data set of 10 lung cancer subjects.

In some embodiments, during a fraction, two subject protocols are used. A first subject protocol is loaded and used to acquire CBCT images. After the CBCT images are acquired, the first subject protocol is closed. The first subject protocol includes a non-adaptive treatment plan. After the treatment plan is adapted, a second subject protocol including the adapted treatment plan is loaded to system 100 and the adapted treatment plan is delivered. As such, use of the non-adapted treatment plan is prevented by closing the first patient protocol and afterwards loading the second patient protocol.

Unlike known adaptive system 200, adaptive radiotherapy computing device 120 may be separate from existing workstation 116. As a result, the functionalities of system 100 is improved without significant changes to system 100, minimizing the cost of upgrading.

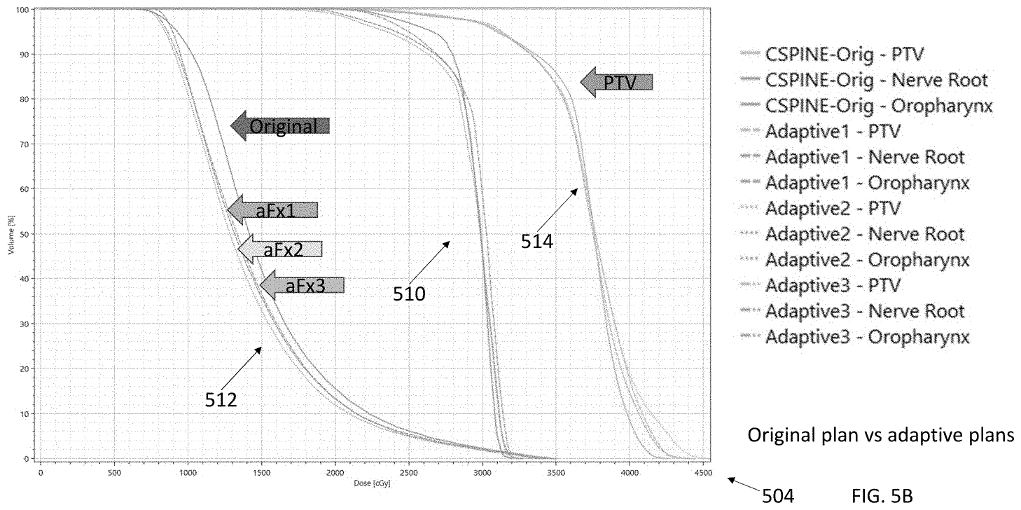

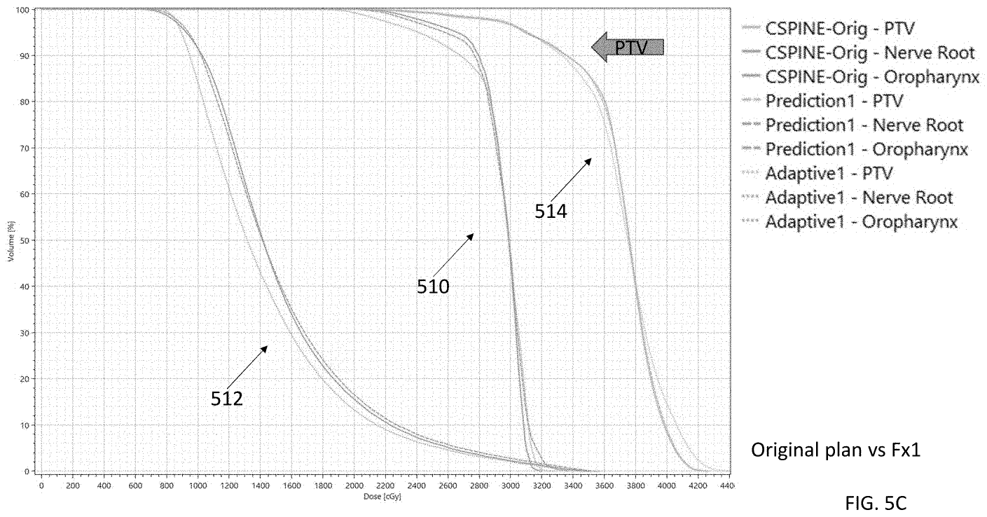

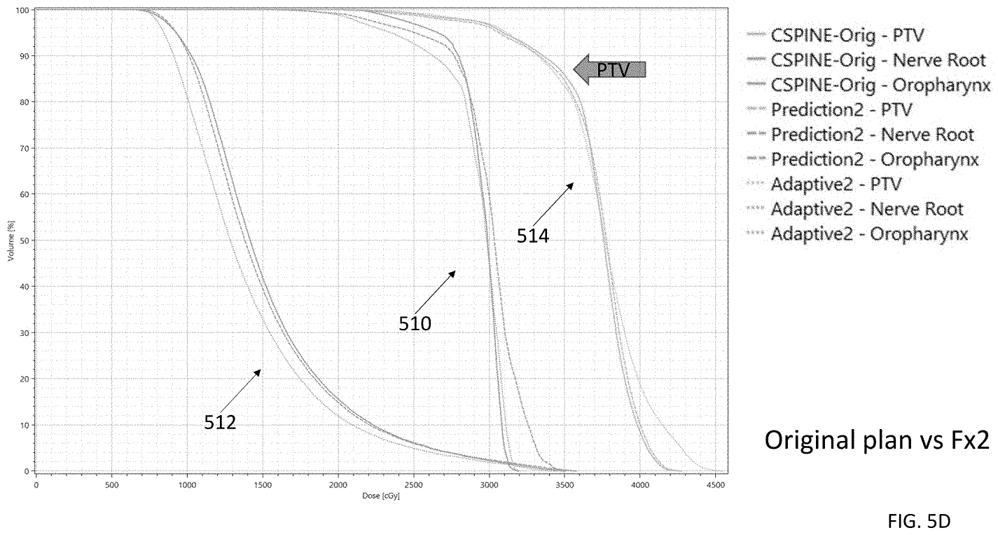

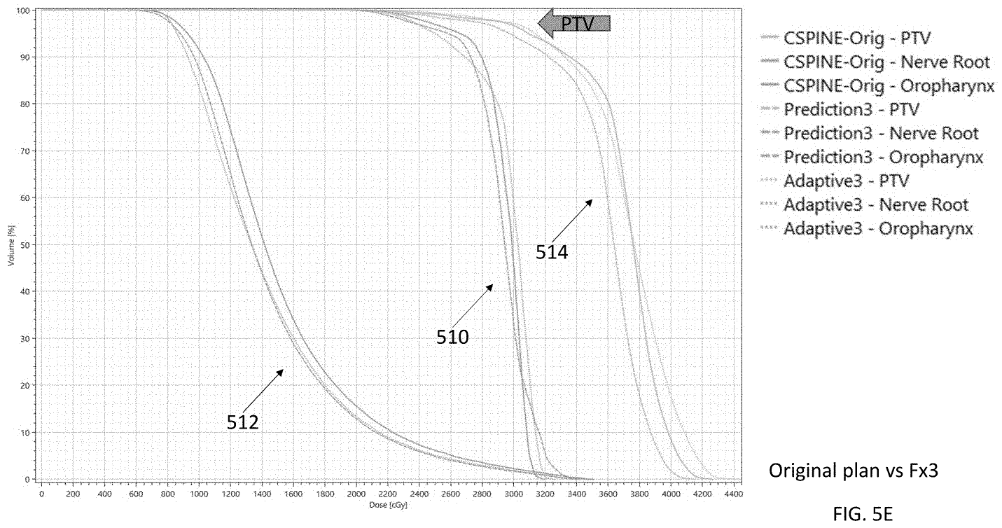

FIGS. 5A-5E show treatment plans. In FIGS. 5A-5E, the x-axis is the dosage, and the y-axis is the volume of the tissue. FIG. 5A shows a nonadaptive treatment plan 502 without the systems and methods described herein. FIG. 5B shows an adaptive treatment plan 504 with the systems and methods described herein. FIGS. 5C-5E shows comparisons between nonadaptive treatment plan 502 and adaptive treatment plan 504. The original plan (pointed with arrow Original) is the treatment plan for day 0. Prediction plans 508 (pointed with arrow pFx1, pFx2, or pFx3) are the treatment plan for fractions of the treatment under nonadaptive treatment plan 502 (FIG. 5A). Adaptive plans 509 (pointed with arrows aFx1, aFx2, or aFx3) are the treatment plan for fractions of the treatment under adaptive treatment plan 504 (FIG. 5B). For example, prediction 1 (pointed to by arrows pFx1) is the treatment plan of fraction 1 or day 1 in nonadaptive treatment plan 502, prediction 2 (pointed with arrow pFx2) is for fraction 2, and prediction 3 (pointed with pFx3) is for fraction 3. Adaptive 1 (pointed to by arrows aFx1) is the treatment plan of fraction 1 in adaptive treatment plan 504, adaptive 2 (pointed with arrow aFx2) is for fraction 2, and adaptive 3 (pointed with aFx3) is for fraction 3. FIG. 5A shows the original plan and prediction plans of three groups of tissues, group 510 of nerve, group 512 of oropharynx, and group 514 of tumor. Groups 510 and 512 are healthy tissues, damage to which should be avoided or limited. As shown in FIG. 5A, prediction plans reduce doses on tumor while keeping doses towards healthy tissue constant across fractions, causing damage to healthy tissue while reducing the effects on tumor. As a result, the treatment efficacy is not optimized and unwanted damages may be introduced. In contrast, as shown in FIGS. 5B-5E, adaptive treatment plans 504 provide relatively constant doses towards the tumor while reducing doses toward healthy tissue as treatment progresses, optimizing the efficacy of the treatment and avoiding unwanted damages to the subject.

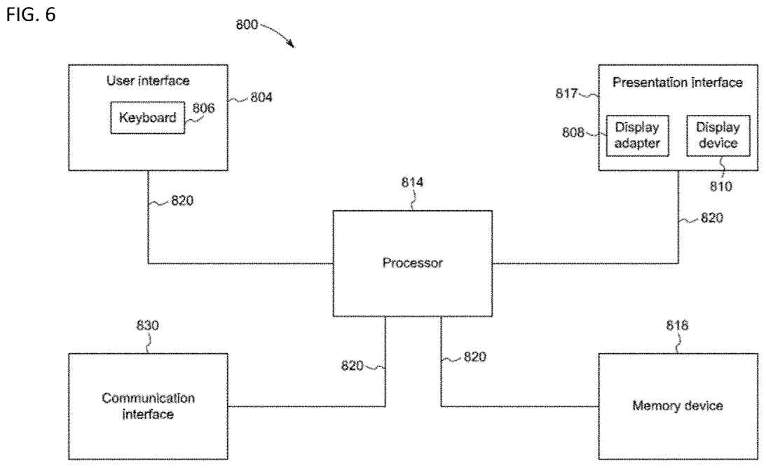

Workstation 116 and adaptive radiotherapy computing device 120 described herein may be any suitable computing device 800 and software implemented therein. FIG. 6 is a block diagram of an exemplary computing device 800. In the exemplary embodiment, computing device 800 includes a user interface 804 that receives at least one input from a user. User interface 804 may include a keyboard 806 that enables the user to input pertinent information. User interface 804 may also include, for example, a pointing device, a mouse, a stylus, a touch sensitive panel (e.g., a touch pad and a touch screen), a gyroscope, an accelerometer, a position detector, and/or an audio input interface (e.g., including a microphone).

Moreover, in the exemplary embodiment, computing device 800 includes a presentation interface 817 that presents information, such as input events and/or validation results, to the user. Presentation interface 817 may also include a display adapter 808 that is coupled to at least one display device 810. More specifically, in the exemplary embodiment, display device 810 may be a visual display device, such as a cathode ray tube (CRT), a liquid crystal display (LCD), a light-emitting diode (LED) display, and/or an “electronic ink” display. Alternatively, presentation interface 817 may include an audio output device (e.g., an audio adapter and/or a speaker) and/or a printer.

Computing device 800 also includes a processor 814 and a memory device 818. Processor 814 is coupled to user interface 804, presentation interface 817, and memory device 818 via a system bus 820. In the exemplary embodiment, processor 814 communicates with the user, such as by prompting the user via presentation interface 817 and/or by receiving user inputs via user interface 804. The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are exemplary only, and thus are not intended to limit in any way the definition and/or meaning of the term “processor.”

In the exemplary embodiment, memory device 818 includes one or more devices that enable information, such as executable instructions and/or other data, to be stored and retrieved. Moreover, memory device 818 includes one or more computer readable media, such as, without limitation, dynamic random access memory (DRAM), static random access memory (SRAM), a solid state disk, and/or a hard disk. In the exemplary embodiment, memory device 818 stores, without limitation, application source code, application object code, configuration data, additional input events, application states, assertion statements, validation results, and/or any other type of data. Computing device 800, in the exemplary embodiment, may also include a communication interface 830 that is coupled to processor 814 via system bus 820. Moreover, communication interface 830 is communicatively coupled to data acquisition devices.

In the exemplary embodiment, processor 814 may be programmed by encoding an operation using one or more executable instructions and providing the executable instructions in memory device 818. In the exemplary embodiment, processor 814 is programmed to select a plurality of measurements that are received from data acquisition devices.

In operation, a computer executes computer-executable instructions embodied in one or more computer-executable components stored on one or more computer-readable media to implement aspects of the invention described and/or illustrated herein. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

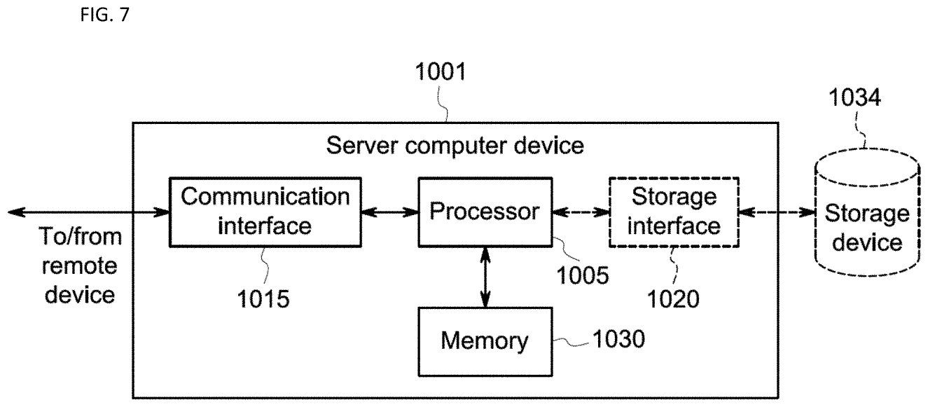

FIG. 7 illustrates an exemplary configuration of a server computer device 1001 such as adaptive radiotherapy computing device 120. Server computer device 1001 also includes a processor 1005 for executing instructions. Instructions may be stored in a memory area 1030, for example. Processor 1005 may include one or more processing units (e.g., in a multi-core configuration).

Processor 1005 is operatively coupled to a communication interface 1015 such that server computer device 1001 is capable of communicating with a remote device or another server computer device 1001. For example, communication interface 1015 may receive data from workstation 116, via the Internet.

Processor 1005 may also be operatively coupled to a storage device 1034. Storage device 1034 is any computer-operated hardware suitable for storing and/or retrieving data, such as, but not limited to, wavelength changes, temperatures, and strain. In some embodiments, storage device 1034 is integrated in server computer device 1001. For example, server computer device 1001 may include one or more hard disk drives as storage device 1034. In other embodiments, storage device 1034 is external to server computer device 1001 and may be accessed by a plurality of server computer devices 1001. For example, storage device 1034 may include multiple storage units such as hard disks and/or solid state disks in a redundant array of inexpensive disks (RAID) configuration. storage device 1034 may include a storage area network (SAN) and/or a network attached storage (NAS) system.

In some embodiments, processor 1005 is operatively coupled to storage device 1034 via a storage interface 1020. Storage interface 1020 is any component capable of providing processor 1005 with access to storage device 1034. Storage interface 1020 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 1005 with access to storage device 1034.

At least one technical effect of the systems and methods described herein includes (a) providing adaptive radiotherapy to radiotherapy systems having a C-arm gantry; (b) flexible adaptation of treatment plans; (c) cost-effective improvements in functionalities of radiotherapy systems by using existing contouring and planning modules in the radiotherapy systems.

Exemplary embodiments of systems and methods of adaptive radiotherapy are described above in detail. The systems and methods are not limited to the specific embodiments described herein but, rather, components of the systems and/or operations of the methods may be utilized independently and separately from other components and/or operations described herein. Further, the described components and/or operations may also be defined in, or used in combination with, other systems, methods, and/or devices, and are not limited to practice with only the systems described herein.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A linear particle accelerator (LINAC) radiotherapy system of a subject, comprising:
a gantry, comprising:
a radiation delivery assembly including LINACs and configured to generate radiation; and
an X-ray imaging assembly configured to image a subject,
wherein the gantry defines a C-arm; and
an adaptive radiotherapy computing device, the adaptive radiotherapy computing device comprising at least one processor in communication with at least one memory device, and the at least one processor programmed to:
receive first images of the subject acquired by an imaging system,
receive second images of the subject acquired by the X-ray imaging assembly; wherein the first images have higher resolutions than the second images;
adapt a treatment plan using the second images, wherein the treatment plan was designed based on the first images, and a level of optimization in adapting the treatment plan is adjustable; and
output the adapted treatment plan.

2. The system of claim 1, wherein the at least one processor is further programmed to:
adapt contours in the treatment plan by registering the first images with the second images.

3. The system of claim 2, wherein the at least one processor is further programmed to:
adapt the treatment plan based on the adapted contours.

4. The system of claim 1, wherein the at least one processor is further programmed to:
adapt the treatment plan using existing modules of the system that are separate from the adaptive radiotherapy computing device.

5. The system of claim 1, wherein the at least one processor is further programmed to:
adapt the treatment plan using prior knowledge.

6. The system of claim 1, wherein the at least one processor is further programmed to:
adapt the treatment plan while the subject is positioned in the system.

7. The system of claim 1, wherein the at least one processor is further programmed to:
perform a quality assurance of the treatment plan.

8. An adaptive radiotherapy computing device of a linear particle accelerator (LINAC) radiotherapy system, the adaptive radiotherapy computing device comprising at least one processor in communication with at least one memory device, and the at least one processor programmed to:
receive first images of a subject acquired by an imaging system,
receive second images of the subject acquired by an X-ray imaging assembly in a C-arm gantry of a LINAC radiotherapy system, wherein the first images have higher resolutions than the second images;
adapt a treatment plan using the second images, wherein the treatment plan was designed based on the first images, and a level of optimization in adapting the treatment plan is adjustable; and
output the adapted treatment plan.

9. The adaptive radiotherapy computing device of claim 8, wherein the at least one processor is further programmed to:
adapt contours in the treatment plan by registering the first images with the second images.

10. The adaptive radiotherapy computing device of claim 9, wherein the at least one processor is further programmed to:
adapt the treatment plan based on the adapted contours.

11. The adaptive radiotherapy computing device of claim 8, wherein the at least one processor is further programmed to:
adapt the treatment plan using existing modules of the LINAC radiotherapy system that are separate from the adaptive radiotherapy computing device.

12. The adaptive radiotherapy computing device of claim 8, wherein the at least one processor is further programmed to:
adapt the treatment plan using prior knowledge.

13. The adaptive radiotherapy computing device of claim 8, wherein the at least one processor is further programmed to:
adapt the treatment plan while the subject is positioned in the system.

14. The adaptive radiotherapy computing device of claim 8, wherein the at least one processor is further programmed to:
perform a quality assurance of the treatment plan.

15. A method of adapting radiotherapy on a subject with a linear particle accelerator (LINAC) radiotherapy system, comprising:
receiving first images of a subject acquired by an imaging system,
receiving second images of the subject acquired by an X-ray imaging assembly in a C-arm gantry of a LINAC radiotherapy system, wherein the first images have higher resolutions than the second images;
adapting a treatment plan using the second images, wherein the treatment plan was designed based on the first images, wherein adapting a treatment plan further comprises adjusting a level of optimization in adapting the treatment plan; and
outputting the adapted treatment plan.

16. The method of claim 15, wherein adapting a treatment plan further comprises adapting contours in the treatment plan by registering the first images with the second images.

17. The method of claim 16, wherein adapting a treatment plan further comprises:
adapting the treatment plan based on the adapted contours.

18. The method of claim 15, wherein adapting a treatment plan further comprises:
adapting the treatment plan using existing modules of the LINAC radiotherapy system.

19. The method of claim 15, wherein adapting a treatment plan further comprises:
adapting the treatment plan using prior knowledge.

20. The method of claim 15, wherein adapting a treatment plan further comprises:
adapting the treatment plan while the subject is positioned in the system.
